(19) United States
(12) Patent Application Publication
(10) Pub. No.: US 2024/0052385 A1
(43) Pub. Date: Feb. 15, 2024

(54) METHOD FOR INCREASING YIELD OF EICOSAPENTAENOIC ACID IN SCHIZOCHYTRIUM SP.

(71) Applicants: SOUTH CHINA UNIVERSITY OF TECHNOLOGY, Guangzhou (CN); YHSN Technology Ltd., Guangzhou (CN)

(72) Inventors: Yonghua WANG, Guangzhou (CN); Shuxin MO, Guangzhou (CN); Zheng XIAO, Guangzhou (CN); Dongming LAN, Guangzhou (CN); Changmei LEI, Guangdong (CN)

(21) Appl. No.: 18/551,667

(22) PCT Filed: Oct. 18, 2021
(86) PCT No.: PCT/CN2021/124344

§ 371 Date: Sep. 21, 2023

(30) Foreign Application Priority Data
Mar. 22, 2021 (CN) 202110303550.0

(51) Int. Cl. C12P 7/6432; C12N 1/12

(52) U.S. Cl. CPC C12P 7/6432; C12N 1/125; C12R 2001/89

(57) ABSTRACT

A method for increasing the yield of eicosapentaenoic acid in Schizochytrium sp., the method comprising: inoculating Schizochytrium sp. ATCC 20888 into a fermentation culture medium, fermenting same under an aerobic condition, changing the temperature when fermenting is performed to the middle of a logarithmic phase, continuing fermenting same, and controlling the dissolved oxygen (DO) value to be 2%-10% after changing the temperature, wherein changing the temperature increases the initial fermentation temperature to 32° C.-37° C. from 25° C.-30° C. EPA is produced by means of fermenting with Schizochytrium sp. ATCC 20888, the dry weight of thalli in the obtained fermentation liquor reaches 66.15 g/L, the yield of oil is 9.97 g/L, and EPA accounts for 13.33% of fatty acid.

### TECHNICAL FIELD

The present invention belongs to a field of fermentation, and relates to a method for increasing the yield of eicosapentaenoic acid in Schizochytrium sp.

### BACKGROUND

Long-chain polyunsaturated fatty acids (LC-PUFA) refer to straight-chain fatty acids with two or more double bonds and a carbon chain length of 18-22 carbon atoms. LC-PUFA, like vitamins and mineral elements, is an essential nutrient for human body and a substance with important medical and healthcare functions. LC-PUFAs may be classified into omega-3 and omega-6 polyunsaturated fatty acids. Among the omega-3 polyunsaturated fatty acids, docosahexaenoic acid (DHA) and eicosapentaenoic acid (EPA) are most important. The omega-3 polyunsaturated fatty acids have a prominent effect on maintaining the health function of heart, angiocarpy, kidneys, and brain, and preventing obesity and metabolism syndrome, cardiovascular diseases, inflammation, neurodegenerative diseases, and other diseases. Insufficient intake of omega-3 polyunsaturated fatty acids for a long time easily leads to dysfunction of important organs such as heart and brain etc. With the rising standard of living, people are in pursuit of a more healthy life and the global demand for omega-3 polyunsaturated fatty acids will increase year by year. It is estimated that the global demand for omega-3 polyunsaturated fatty acids will increase by 16% every year, from 2015 to 2025. 99% DHA has a market price of 144 dollars per gram, while 99% EPA has a market price of 2,000 dollars per gram, much higher than that of DHA.

Eicosapentaenoic acid (EPA) is an omega-3 polyunsaturated fatty acid that is extremely important to the human body, and has important physiological functions in prevention and treatment of cardiovascular diseases, treatment of schizophrenia and depressive disorder, anti-inflammation, and anti-cancer etc. At present, EPA has a promising commercial prospect in industries such as healthcare food, medicine, feed, and the like. A traditional source of EPA is fish oil. However, due to effects of exhaustion of marine resources and environmental pollution, the quality and the yield of the fish oil are unsatisfactory, which cannot meet the increasing demand of people. Therefore, it is currently an urgent issue to find an environment-friendly way capable of sustainable production.

Schizochytrium sp. is a heterotrophic marine fungus, which contains a large amount of DHA (40%-60%). EPA also exists in the fatty acids of Schizochytrium sp., however, the percentage of EPA among total fatty acids (TFAs) is usually less than 1%, so Schizochytrium sp. is mainly used for producing DHA by fermentation. Because of fast growth rate and high yield, Schizochytrium sp. is usually regarded as a satisfactory sustainable resource for producing DHA, and is one of the microalgaes approved in various countries all over the world for commercial production of DHA, which has promising commercial prospects. Due to the lower content of EPA in Schizochytrium sp., current research on Schizochytrium sp. is mainly focused on the selection and breeding of superior strains, the biosynthesis and the optimization of fermentation conditions of DHA, and there are fewer researches on the preparation of EPA by using fermentation of Schizochytrium sp.

Ling Xueping et al. increased the percentage of EPA among total fatty acids from 0.45% to 0.65% by adding 50 mg/L fluridone when Schizochytrium sp. was cultured for 24 hours (Ling Xueping, Li Jun, and Lu Yinghua, et al. Regulation and Control Method for Increasing EPA Content in Schizochytrium sp. and application thereof). Meng Tong increased the percentage of EPA among total fatty acids from 0.58% to 0.98% by supplementing materials for fermentation in batches and adding inorganic salts for fermentation for 168 hours during the fermentation (Meng Tong, Research on Fermentation Technology for Producing Omega-3 Polyunsaturated Fatty Acids from Schizochytrium sp. [D]. Xiamen University, 2019).

The current research on EPA production from Schizochytrium sp. has the problems of a low percentage of EPA among total fatty acids, a long fermentation time, and toxicity of exogenous additives etc. Therefore, the key of replacing the extraction of EPA from fish oil by fermentation of Schizochytrium sp. is to further increase the yield of EPA by optimizing fermentation processes.

### SUMMARY

Based on improvements to the existing fermentation technology, the present invention provides a method for increasing the yield of eicosapentaenoic acid in Schizochytrium sp., which aims at increasing a synthesis amount of eicosapentaenoic acid by means of changing a fermentation temperature and moderately controlling dissolved oxygen, further increasing the yield of eicosapentaenoic acid.

A purpose of the present invention is implemented by the following technical solutions.
(1) Strain: Schizochytrium sp. ATCC 20888 purchased from American Type Culture Collection.
(2) Culture media:

A solid culture medium (g/L) comprises glucose 30-50, yeast extract powder 8-10, sodium glutamate 10-30, magnesium sulfate 3-5, ammonium sulfate 1.5-3, sodium sulfate 20-40, potassium dihydrogen phosphate 2-5, potassium chloride 0.5-1.5, trace element solution 1.5-3 mL, and agar powder 15-20.

A seed culture medium (g/L) comprises glucose 50-80, yeast extract powder 8-10, sodium glutamate 40-80, magnesium sulfate 3-5, ammonium sulfate 1.5-3, sodium sulfate 20-40, potassium dihydrogen phosphate 2-5, potassium chloride 0.5-1.5, and trace element solution 1.5-3 mL.

A fermentation culture medium (g/L) comprises glucose 100-120, yeast extract powder 5-8, sodium glutamate 20-60, magnesium sulfate 3-5, ammonium sulfate 3-5, sodium sulfate 20-40, potassium dihydrogen phosphate 2-5, potassium chloride 0.5-1.5, and trace element solution 1.5-3 mL.

(3) A formula of the trace element solution (g/L) comprises disodium EDTA 5-8, cobalt chloride 0.005-0.02, manganese chloride 0.5-1, zinc sulfate 1-3, ferrous sulfate 0.05-1, copper sulfate 0.5-1, sodium molybdate 0.005-0.02, and nickel sulfate 0.05-1.

(4) Cell density: using an ultraviolet spectrophotometer with a wavelength of 600 nm; collecting a sample and diluting it appropriately, where a determination range is 0.2-0.8; multiplying a dilution multiple by a determined value; and repeating the process for three times.

(5) DCW determination: taking 10 mL of a fermentation solution, and centrifuging it at 5000 g for 5 min, then pouring supernatant, washing the strain twice with deionized water to obtain a wet strain of Schizochytrium sp.; drying the wet strain in a drying oven at 80° C., and weighing a weight of the dry strain until a constant weight is obtained; and repeating the process for three times.

(6) Determination of total fatty acids: taking 10 mL of the fermentation solution, centrifuging it at 5000 g for 5 min, and washing the strain twice with deionized water; adding 5 mL of hydrochloric acid into the wet strain, swirling for 2 min, heating for 1 h in a water bath at 80° C., and extracting for three times with n-hexane until supernatant is transparent; completely dissolving an oil sample in a n-hexane solution, performing rotary evaporation to recover a solvent, drying the solvent, and weighing the oil; performing a methyl esterification operation for 30 min at 60° C. by using 3 mL of methanol containing 2% sodium hydroxide, and detecting the obtained fatty acid methyl ester with a gas chromatograph-mass spectrometer, with a gas chromatographic column being CP-SiL88, Helium being used as a carrier gas, a split stream sampling way being used, a temperature increasing procedure of the chromatographic column being: an initial temperature of 140° C., this temperature being maintained ° C. for 5 min, then increasing the temperature to 220° C. at a rate of 10° C./min, maintaining this temperature for 17 min A content of total fatty acids and a content of each fatty acid are calculated by adopting a peak-area normalization method, according to an internal standard.

Schizochytrium sp. ATCC 20888 is inoculated into a fermentation culture medium and is fermented under an aerobic condition. The temperature is changed when fermenting to the middle of a logarithmic phase, and the fermentation continues. A dissolved oxygen (DO) value is controlled to be 2%-10% after changing the temperature, the step of changing the temperature being to increase an initial fermentation temperature of 25-30° C. to 32-37° C.

Preferably, the fermentation time corresponding to the middle of the logarithmic phase is 24±4 h, the initial fermentation temperature is 28±1° C., and the changed temperature is 34±1° C.

Preferably, the dissolved oxygen (DO) value before changing the temperature is controlled to be 50% or more.

Preferably, the formula of the trace element solution (g/L) contained in the fermentation culture medium comprises: disodium EDTA 5-8, cobalt chloride 0.005-0.02, manganese chloride 0.5-1, zinc sulfate 1-3, ferrous sulfate 0.05-1, copper sulfate 0.5-1, sodium molybdate 0.005-0.02, and nickel sulfate 0.05-1.

Preferably, the fermentation culture medium (g/L) is composed of: glucose 100, yeast extract powder 8, sodium glutamate 40, magnesium sulfate 4.48, ammonium sulfate 1.5, sodium sulfate 37, potassium dihydrogen phosphate 3.5, potassium chloride 1, and trace element solution 2 mL.

Preferably, the content of the glucose is ensured to be maintained at 10-30 g/L during the fermentation.

Preferably, the time for the temperature-changing fermentation reaction is 96 h±4 h.

Preferably, the fermentation conditions are: a pH value of 5.5-7, a ventilation rate of 3-5 L/min, and a rotation speed of 300-700 rpm.

Compared with the prior art, the present invention has the following beneficial effects.

By establishing the method for increasing the yield of eicosapentaenoic acid in Schizochytrium sp., a method for increasing the temperature of the culture medium and controlling the low dissolved oxygen in the middle of the logarithmic phase of the fermentation, the percentage of EPA among total fatty acids in Schizochytrium sp. is increased, the oil yield reaches 9.97 g/L, and the percentage of EPA among total fatty acids reaches 13.33%, so that the oil quality of the Schizochytrium sp. is improved.

### BRIEF DESCRIPTION OF DRAWINGS

FIG. 1 shows growth characteristics of Schizochytrium sp. fermented at 28° C.

FIG. 2 shows growth characteristics of Schizochytrium sp. fermented at 34° C.

FIG. 3 shows growth characteristics of the Schizochytrium sp. fermented at the conditions of 10% DO and 2% DO; FIG. 3a shows growth characteristics of the fermented Schizochytrium sp.; FIG. 3b shows an oil yield of the fermented Schizochytrium sp.; FIG. 3c shows consumption situation of sodium glutamate for fermenting Schizochytrium sp.; and FIG. 3d shows percentages of DHA and EPA among total fatty acids of the fermented Schizochytrium sp.

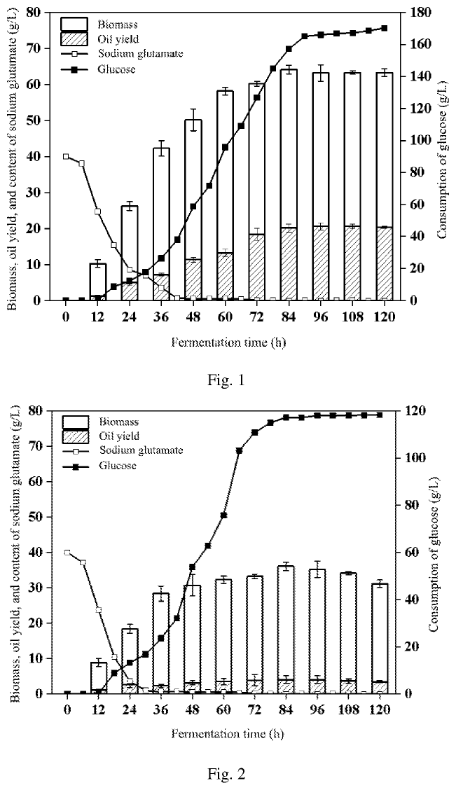

### DETAILED DESCRIPTION OF EMBODIMENTS

The present invention is further described in detail below in combination with specific examples, but embodiments of the present invention are not limited thereto. For the technological parameters that are not particularly specified, reference may be made to the conventional technology.

### Example 1

To study growth characteristics of Schizochytrium sp. at a suitable growth temperature (28° C.) and a high temperature (34° C.), the fermentation under the two temperature conditions was performed respectively in a 5 L fermentation tank.

A shaken seed solution of Schizochytrium sp. was inoculated into the 5 L fermentation tank according to an inoculation amount of 10%, the culture temperature was 28° C. and 34° C. respectively, pH was natural, a ventilation rate was 3 L/min, and a rotation speed was 500 rpm; glucose was added when the glucose content was less than 20 g/L, so that the glucose content was maintained 20 g/L or more; and a fermentation time was 120 hours.

Results show that 28° C. is relatively suitable for the growth of Schizochytrium sp., at the end of fermentation, biomass reaches 63.31 g/L, the oil yield reaches 20.39 g/L, and the percentage of EPA among total fatty acids is 0.86% (Table 1). When Schizochytrium sp. is fermented at 34° C., its growth is limited, upon 120 h fermentation, the biomass is 31.34 g/L, the oil yield is 3.97 g/L, however, the percentage of EPA among total fatty acids is increased significantly, and the percentage of EPA among total fatty acids at the end of fermentation reaches 7.17% (Table 1).

TABLE 1
Changes of fatty acid compositions of Schizochytrium sp. at 28° C. and 34° C.

| Percentage | | | | | | | | | | |
|---|---|---|---|---|---|---|---|---|---|---|
| content of | | | | | | | | | | |
| fatty acid | 28° C. | | | | | 34° C. | | | | |
| (%) | 24 h | 48 h | 72 h | 96 h | 120 h | 24 h | 48 h | 72 h | 96 h | 120 h |
| C14:0 | 9.08 | 5.46 | 5.26 | 7.61 | 7.41 | 7.63 | 6.50 | 4.31 | 3.80 | 1.81 |
| C16:0 | 35.16 | 17.36 | 17.72 | 18.71 | 19.39 | 29.02 | 25.29 | 18.25 | 14.20 | 11.76 |
| C18:0 | 1.49 | 0.55 | 0.57 | 0.82 | 0.98 | 0.859 | 0.78 | 0.52 | 0.52 | 0.47 |
| EPA | 0.23 | 0.54 | 0.66 | 0.78 | 0.86 | 1.147 | 1.14 | 2.13 | 4.75 | 7.17 |
| Omega-6- | 8.53 | 14.93 | 15.42 | 12.52 | 13.08 | 12.55 | 12.98 | 11.38 | 15.90 | 17.60 |
| DPA | | | | | | | | | | |
| Omega-3- | — | 0.66 | 0.84 | 1.43 | 1.48 | 1.23 | 1.16 | 1.34 | 2.04 | 2.53 |
| DPA | | | | | | | | | | |
| DHA | 43.75 | 45.71 | 42.41 | 35.61 | 36.89 | 45.36 | 39.61 | 39.98 | 40.33 | 43.94 |

### Example 2

To study the influence of a dissolved oxygen condition on Schizochytrium sp., a growth situation under different dissolved oxygen (DO) (10% and 2%) conditions was studied respectively, and the optimization of the two dissolved oxygen conditions was performed in a 5 L fermentation tank respectively.

The shaken seed solution of Schizochytrium sp. was inoculated into the 5 L fermentation tank according to an inoculation amount of 10%, a culture temperature was 28° C., pH was natural, a ventilation rate was 3 L/min, and an initial rotation speed was 500 rpm; after the dissolved oxygen decreased to 50%, the rotation speed and the ventilation rate were adjusted to maintain the dissolved oxygen (DO) value at 50% in the former 24 h, and maintain the dissolved oxygen value at 10% and 2% respectively in the late 96 h; glucose was added when the glucose content was less than 20 g/L, so that the glucose content was maintained 20 g/L or more; and a fermentation time was 120 hours.

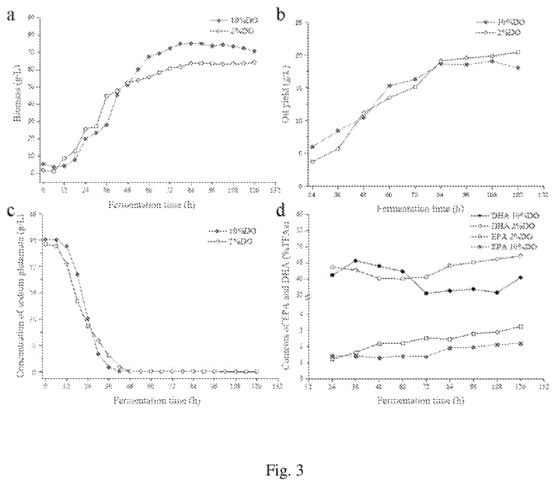

The growth characteristics of Schizochytrium sp. under different dissolved oxygen conditions are shown as FIG. 3. Compared with the condition of 2% DO, cells grow better under the condition of 10% DO, the maximal biomass reaches 75.067 g/L (FIG. 3a), the consumption of sodium glutamate at 36 h is faster (FIG. 3c), but the oil yield (FIG. 3b), and the percentage contents of DHA and EPA among total fatty acids are less than those under the condition of 2% DO (FIG. 3d). The biomass of Schizochytrium sp. under the condition of 2% DO continues to increase during the fermentation, and the maximal biomass reaches 61.21 g/L, and the oil yield and the percentage content of EPA among total fatty acids reaches 20.39 g/L and 3.29% respectively. The results show that the cells grow better at a high dissolved oxygen level, while the low dissolved oxygen level has a positive influence on the accumulation of the oil and EPA.

### Example 3

Optimization of different temperature-changing time nodes: to study the influence of the temperature-changing time node on Schizochytrium sp., the growth situations of Schizochytrium sp. at different temperature-changing time nodes (24 h, 48 h, and 72 h) were studied, and the three temperature-changing time nodes were optimized respectively in the 5 L fermentation tank. The experiment was divided into a 24-hour temperature-changing group, a 48-hour temperature-changing group, and a 72-hour temperature-changing group. After fermentation for 24 h (the middle of the logarithmic phase), 48 h (a preliminary stage of a stable phase), and 72 h (the middle of the stable phase), the temperature was changed and increased to 34° C. The 28° C. constant-temperature fermentation was taken as a control group. The shaken seed solution of Schizochytrium sp. was inoculated into the 5 L fermentation tank according to an inoculation amount of 10%, a culture temperature was respectively 28° C., pH was natural, a ventilation rate was 3 L/min, and a rotation speed was 500 rpm. Fermentation results are shown as Table 2.

TABLE 2
Influence of different temperature-changing time on biomass,
oil yield and percentage content of EPA, and EPA yield of
Schizochytrium sp.

| | | 24-hour | 48-hour | 72-hour |
|---|---|---|---|---|
| | | temperature- | temperature- | temperature- |
| | Control | changing | changing | changing |
| | group: | group | group | group |
| Biomass | 63.31 | 43.17 | 53.83 | 68.44 |
| (g/L) | | | | |
| Oil yield | 20.39 | 10.23 | 10.55 | 20.10 |
| (g/L) | | | | |
| EPA content | 0.86 | 7.93 | 4.93 | 2.31 |
| (% TFAs) | | | | |
| EPA yield | 0.17 | 0.78 | 0.50 | 0.46 |
| (g/L) | | | | |

The results show that the later the temperature-changing time is, the smaller the influence on the growth of Schizochytrium sp is. When the temperature is changed at 72 h, the growth of Schizochytrium sp. is substantially not influenced. In the aspect of oil accumulation, the high-temperature condition may reduce the oil accumulation of Schizochytrium sp. At the end of 24-hour temperature-changing fermentation, the EPA content (% TFAs) is maximal, and the percentage content of EPA among total fatty acids is 7.93%, which is increased by 9.22 times compared with that of the constant-temperature fermentation. In conclusion, the 24-hour temperature change is the optimal condition.

### Example 4

The temperature of Schizochytrium sp. was changed at 28° C. in the middle of the logarithmic phase (24 h), and the temperature was increased to 34° C.; an initial ventilation rate was 3 L/min, an initial rotation speed was 500 rpm, pH was natural, after the dissolved oxygen decreased to 50%, the rotation speed and the ventilation rate were adjusted to maintain the dissolved oxygen at 50% in the former 24 h, and maintain the dissolved oxygen at 2% in the late 96 h, and the accumulation of EPA in Schizochytrium sp. was induced by high temperature and low dissolved oxygen conditions. Experimental results are compared with data in the Example 1, seeing Table 4.

Results show that, as seen from Table 3, under this strategy condition, when the preliminary fermentation temperature is controlled at 28° C., high cell density may be ensured; upon fermentation for 24 h, the temperature is increased to 34° C., which promotes the accumulation of EPA, the percentage content of EPA among total fatty acids is increased significantly, and at the end of fermentation, the percentage content of EPA is increased to 13.33%.

TABLE 3
Changes of biomass, oil yield and several main fatty acids of Schizochytrium sp. under
temperature-changing conditions

| Parameter/fermentation time | 24 h | 48 h | 72 h | 96 h | 120 h |
|---|---|---|---|---|---|
| Biomass (g/L) | 44.02 ± 0.39 | 80.86 ± 0.03 | 94.02 ± 0.65 | 84.68 ± 1.12 | 66.15 ± 0.13 |
| Oil yield (g/L) | 2.46 ± 0.06 | 10.36 ± 0.17 | 13.85 ± 0.13 | 11.70 ± 0.66 | 9.97 ± 0.34 |
| C16:0 content (% TFAs) | 35.98 ± 0.89 | 28.78 ± 0.54 | 24.83 ± 0.08 | 21.09 ± 0.54 | 22.96 ± 0.14 |
| EPA content (% TFAs) | 1.13 ± 0.06 | 3.92 ± 0.03 | 7.83 ± 0.87 | 10.96 ± 0.34 | 13.33 ± 0.16 |
| DPA-Omega-3 content (% TFAs) | 1.26 ± 0.09 | 1.59 ± 0.05 | 2.56 ± 0.11 | 5.49 ± 0.10 | 7.17 ± 0.43 |
| DHA content (% TFAs) | 44.51 ± 0.55 | 45.18 ± 0.34 | 43.50 ± 0.51 | 46.07 ± 0.12 | 45.78 ± 0.18 |

It may be seen from Table 4 that when the fermentation is performed under the optimized process, the biomass and the percentage content of EPA among total fatty acids of Schizochytrium sp. are significantly greater than that of the ordinary fermentation process; and under the strategies of phased temperature control and low dissolved oxygen, the biomass of Schizochytrium sp. reaches 66.15 g/L upon fermentation for 120 h, the oil yield reaches 9.97 g/L, and the percentage content of EPA among total fatty acids reaches 13.33%.

TABLE 4
Comparison of EPA fermentation parameters of Schizochytrium
sp. under different temperature control strategies

| | Experiment group | | | |
|---|---|---|---|---|
| | | | Example 4 | |
| | | | Phased | |
| | | | temperature | |
| | | | control and | |
| | | | low dissolved | Increase |
| | Example 1 | | oxygen | rateª |
| Parameter | 28° C. | 34° C. | strategy | (%) |
| Biomass (g/L) | 63.31 | 31.34 | 66.15 | 4.49 |
| Oil yield (g/L) | 20.39 | 3.97 | 9.97 | — |
| EPA content | 0.86 | 7.17 | 13.33 | 85.91 |
| (% TFAs) | | | | |
| EPA yield (g/L) | 0.17 | 0.27 | 1.28 | 374.07 |
| DH content | 36.90 | 43.96 | 45.78 | 4.14 |
| (% TFAs) | | | | |
| DHA yield (g/L) | 7.22 | 1.67 | 4.38 | |

^{a}comparison with the optimal result under the fermentation conditions of 28° C. and 34° C. in Example 1.

The above-described examples are preferred examples of the present invention, but embodiments of the present invention are not limited by the above-described examples, and any other changes, modifications, substitutions, combinations, simplification, etc. made without departing from the spirit and principle of the present invention should all be equivalent replacement modes, and should all be included in the protection scope of the present invention.

1. A method for increasing the yield of eicosapentaenoic acid in Schizochytrium sp., characterized in that, it comprises: inoculating Schizochytrium sp. into a fermentation culture medium, fermenting it under an aerobic condition, changing the temperature upon fermenting to the middle of a logarithmic phase, continuing to ferment it, and controlling a dissolved oxygen value to be 2%-10% after changing the temperature, with the step of changing the temperature being to increase an initial fermentation temperature of 25-30° C. to 32-37° C.

2. The method for increasing the yield of eicosapentaenoic acid in Schizochytrium sp. according to claim 1, characterized in that, a fermentation time corresponding to the middle of the logarithmic phase is 24±4 h, the initial fermentation temperature is 28±1° C., and the changed temperature is 34±1° C.

3. The method for increasing the yield of eicosapentaenoic acid in Schizochytrium sp. according to claim 1, characterized in that, the dissolved oxygen value before changing the temperature is controlled to be 50% or more.

4. The method for increasing the yield of eicosapentaenoic acid in Schizochytrium sp. according to claim 1, characterized in that, a formula of a trace element solution (g/L) contained in the fermentation culture medium comprises: disodium EDTA 5-8, cobalt chloride 0.005-0.02, manganese chloride 0.5-1, zinc sulfate 1-3, ferrous sulfate 0.05-1, copper sulfate 0.5-1, sodium molybdate 0.005-0.02, and nickel sulfate 0.05-1.

5. The method for increasing the yield of eicosapentaenoic acid in Schizochytrium sp. according to claim 4, characterized in that, the fermentation culture medium (g/L) is composed of: glucose 100, yeast extract powder 8, sodium glutamate 40, magnesium sulfate 4.48, ammonium sulfate 1.5, sodium sulfate 37, potassium dihydrogen phosphate 3.5, potassium chloride 1, and trace element solution 2 mL.

6. The method for increasing the yield of eicosapentaenoic acid in Schizochytrium sp. according to claim 1, characterized in that, the content of glucose is ensured to be maintained at 10-30 g/L during the fermentation.

7. The method for increasing the yield of eicosapentaenoic acid in Schizochytrium sp. according to claim 1, characterized in that, the time for fermentation reaction after changing the temperature is 96 h±4 h.

8. The method for increasing the yield of eicosapentaenoic acid in Schizochytrium sp. according to claim 1, characterized in that, the fermentation conditions are as follows: a pH value of 5.5-7, a ventilation rate of 3-5 L/min, and a rotation speed of 300-700 rpm.

9. The method for increasing the yield of eicosapentaenoic acid in Schizochytrium sp. according to claim 1, characterized in that, Schizochytrium sp. is Schizochytrium sp. ATCC 20888.
